(19) United States
(12) Patent Application Publication
(10) Pub. No.: US 2012/0283124 A1
(43) Pub. Date: Nov. 8, 2012

(54) Cell Surface Marker Expression in Hematopoietic Stem Cells and Progenitors for the Diagnosis, Prognosis, and Treatment of Myelodysplastic Syndromes

(76) Inventors: Christopher Y. Park, New York, NY (US); Wendy W. Pang, Stanford, CA (US); Irving L. Weissman, Stanford, CA (US)

(21) Appl. No.: 13/508,319

(22) PCT Filed: Nov. 9, 2010
(86) PCT No.: PCT/US10/56010

§ 371 Date: Jul. 17, 2012

Related U.S. Application Data
(60) Provisional application No. 61/259,929, filed on Nov. 10, 2009.

(51) Int. Cl. G01N 33/574; C40B 30/04; C12Q 1/68; C12N 5/095

(52) U.S. Cl. 506/9; 435/7.23; 435/325; 435/375; 435/6.13

(57) ABSTRACT

Myelodysplastic syndromes are staged by analysis of the presence of hematopoietic stem and/or progenitor cells, particularly progenitor cells dedicated to the myeloid lineage and hematopoietic stem cells.

### BACKGROUND OF THE INVENTION

The myelodysplastic syndromes (MDS) represent a related group of clonal hematologic disorders characterized by peripheral cytopenias due to ineffective hematopoiesis. The syndromes may arise de novo, or secondarily after treatment with chemotherapy and/or radiation therapy for other diseases. Secondary myelodysplasia usually has a poorer prognosis than does de novo myelodysplasia. MDS transforms to acute myeloid leukemia (AML) in about 30% of patients after various intervals from diagnosis and at variable rates.

MDS occurs predominantly in older patients, though patients as young as 2 years have been reported. Anemia, bleeding, easy bruising, and fatigue are common initial findings. Splenomegaly or hepatosplenomegaly may occasionally be present in association with an overlapping myeloproliferative disorder. Approximately 50% of the patients have a detectable cytogenetic abnormality, most commonly a deletion of all or part of chromosome 5 or 7, or trisomy 8. Although the bone marrow is usually hypercellular at diagnosis, 15% to 20% of patients present with a hypoplastic bone marrow. Hypoplastic myelodysplastic patients tend to have profound cytopenias and may respond more frequently to immunosuppressive therapy.

A variety of risk classification systems have been developed to predict the overall survival of patients with MDS and the evolution from MDS to AML. Clinical variables in these systems have included bone marrow and blood myeloblast percentage, specific cytopenias, age, lactate dehydrogenase level, and bone marrow cytogenetic pattern.

However, diagnosis remains a clinical challenge due to the absence of a single objective criterion for diagnosis, except in the cases where conventional cytogenetics may reveal a clonal abnormality. Therefore, the diagnosis of MDS is often one of exclusion, significantly delaying diagnosis. The development of novel tests to diagnose MDS and to predict which patients will progress to high-risk MDS would be of enormous clinical benefit in order to triage patients to appropriate therapy. It would be particularly beneficial to identify MDS patients at risk of progression to AML.

A quick, accurate and positive diagnosis procedure that is capable of accurately diagnosing MDS is of great interest. The present invention addresses this need.

### SUMMARY OF THE INVENTION

Methods are provided for diagnosis and staging of myelodysplastic syndromes (MDS). In the methods of the invention, hematologic samples, e.g. blood, lymph, bone marrow aspirate, etc. are differentially analyzed for the distribution of hematopoietic stem and progenitor cells among specific phenotypes, which phenotypes include, without limitation, hematopoietic stem cells (HSC); myeloid progenitors; common lymphoid progenitors (CLP); megakaryocyte progenitors; etc. Specifically, it is shown that myelodysplastic syndromes, for example at initial presentation, show reproducible alterations in hematopoietic stem cell and myeloid progenitor cell frequency, with decreased numbers of granulocyte/macrophage progenitors (GMP) and increased numbers of hematopoietic stem cells.

An analysis based on the presence of cell surface markers that provide an assignment of cells into a class of interest, are generally used for determining the distribution of phenotypes in a patient sample. Alterations in the distribution patterns, as compared to a normal patient, are diagnostic of a myelodysplastic syndrome and prognostic for progression of MDS to leukemia. In addition to subset distribution, it has been found that overexpression of CD117 on cells in bone marrow samples is indicative of MDS.

In some embodiments the analysis is performed at initial presentation of a patient, and provides a means of differentiating between MDS and clinical and pathologic mimics of these disorders. It is shown herein that decreasing percentages of granulocyte GMP among myeloid progenitors allows for the detection of MDS with a sensitivity and specificity of at least 80%.

In other embodiments, the analysis provides for determination of the prognosis a patient with established MDS. A prognosis for high risk of progression to acute myelogenous leukemia (AML) is indicated by increasing numbers of CD34+CD38+CD45RA+ cells, even when there is no definite morphologic evidence of increased blasts in bone marrow aspirate smears.

In other embodiments of the invention, it is shown that cell surface markers on hematopoietic stem cells present in MDS bone marrow samples have altered expression, when compared to normal HSC. The differentially expressed markers are useful for selection or elimination of MDS HSC clones; and further allows for the prospective isolation and evaluation of MDS HSC both functionally and molecularly, using in vitro and in vivo assays to test the functional biology and drug susceptibility of MDS HSC. Markers of interest are set forth in Table 1. Markers over-expressed in MDS HSC include, without limitation, CD9, CD69, PTHR2, CD44, CD99, CD200, and CD47. Markers over-expressed in samples of AML/MDS compared to de novo AML blasts, include, without limitation, CD69, IL6R, ITGA6, CF3R, CD44, CD53, and CD55.

### BRIEF DESCRIPTION OF THE DRAWINGS

FIG. 1. MDS bone marrow contains greater frequency of immunophenotypic HSC. A. Representative flow cytometric plots of normal (top) and MDS (bottom) bone marrow samples stained with Lineage, CD34, CD38, CD90, and CD45RA markers, showing gating strategy for HSC and MPP. B. Summary of the frequency of HSC as a percentage of CD34+ cells found in normal and MDS bone marrow samples.

FIG. 2. MDS bone marrow contains fewer immunophenotypic GMP. A.

Representative flow cytometric plots of normal (top) and MDS (bottom) bone marrow samples stained with Lineage, CD34, CD38, CD123, and CD45RA markers, showing gating strategy for CMP, GMP, and MEP. B. Summary of the frequency of GMP as a percentage of total CMP, GMP, and MEP cells found in normal and MDS bone marrow samples.

FIG. 3. Representative examples of cell surface marker profiling of MDS HSC. A.

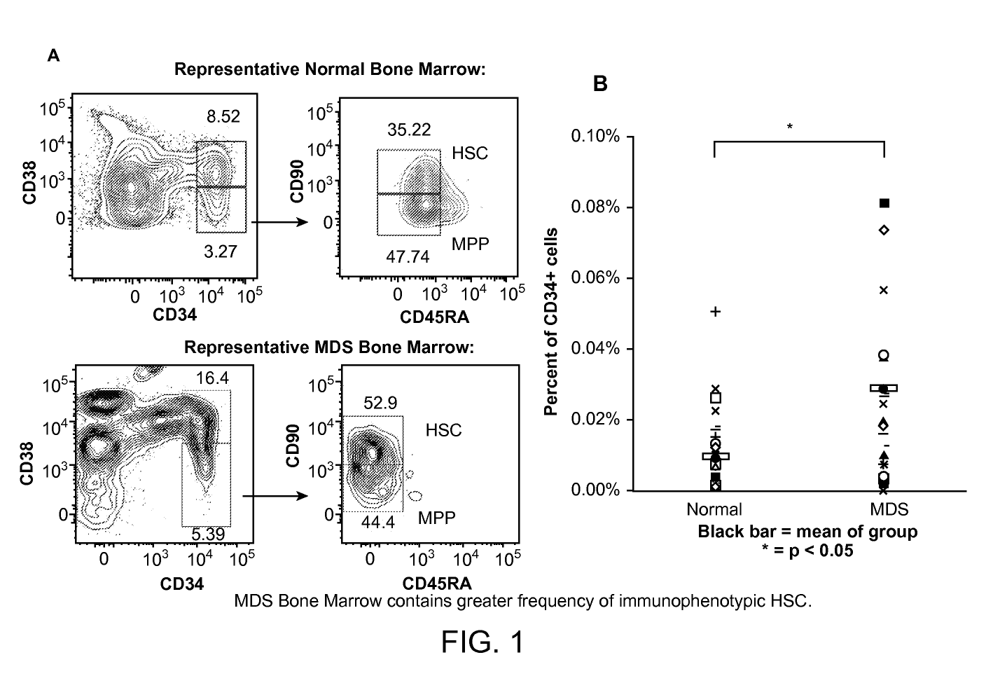

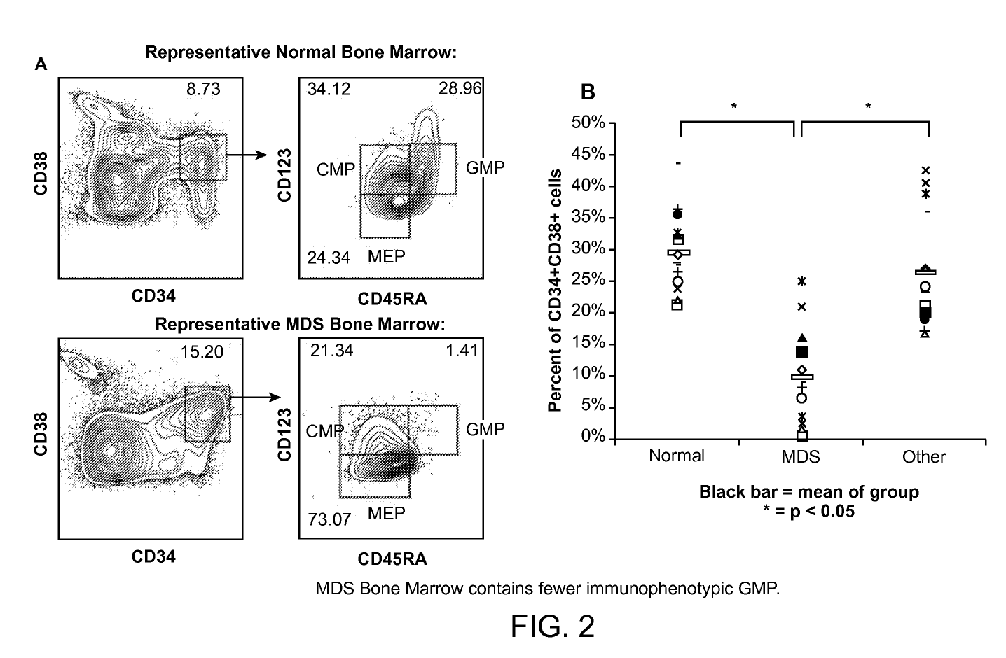

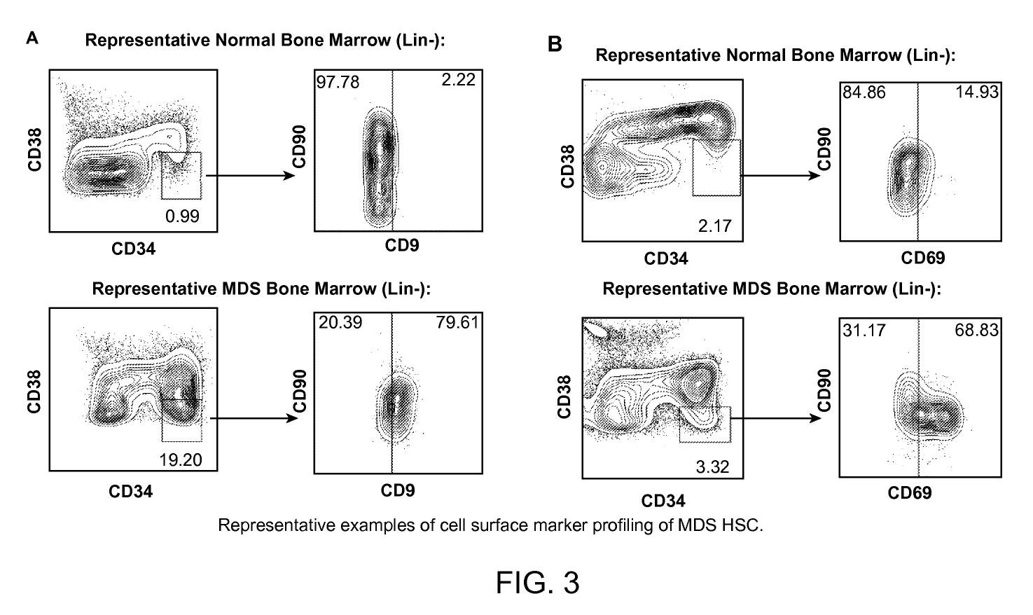

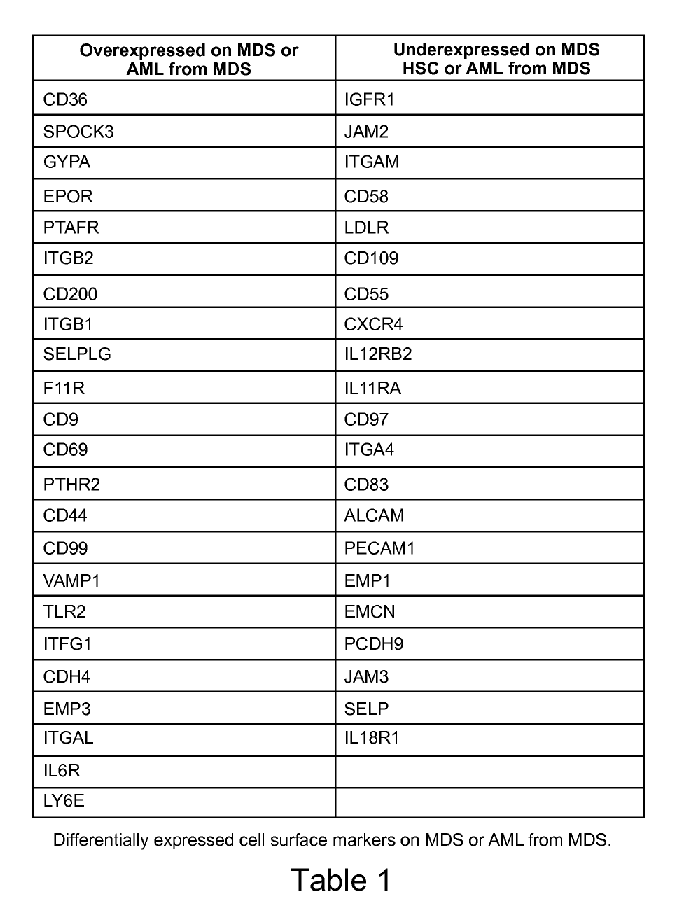

Representative flow cytometric plots of normal (top) and MDS (bottom) bone marrow samples stained with Lineage, CD34, CD38, CD90, and CD9 markers, demonstrating increased expression of CD9 in CD34+CD38− cells from MDS compared to normal. B. Representative flow cytometric plots of normal (top) and MDS (bottom) bone marrow samples stained with Lineage, CD34, CD38, CD90, and CD69 markers, demonstrating increased expression of CD69 in CD34+CD38− cells from MDS compared to normal.

Table 1. Differentially expressed cell surface markers on myelodysplastic cells, or acute myelogenous leukemia arising from MDS.

### DETAILED DESCRIPTION OF THE EMBODIMENTS

Myelodysplastic syndromes are staged by analysis of the presence of hematopoietic stem and/or progenitor cells, particularly progenitor cells dedicated to the myeloid lineage, which progenitor cells include CMP (common myeloid progenitors); megakaryocyte erythroid progenitors (MEP), and myelomonocytic lineages (GMP).

Hematologic samples from a patient suspected of MDS are analyzed for the distribution of hematopoietic stem and progenitor cells among specific phenotypes. Specifically, the distribution of progenitor cells within the myeloid progenitor (MP) class is altered. As used herein, the class of cells, e.g. bone marrow cells, that are considered to be myeloid progenitors are the combined number of progenitor cells that are CMP, GMP and MEP. The myeloid progenitor class can be distinguished from hematopoietic stem cells (HSC) by determining expression of CD34 and CD38, as the HSC are CD34⁺CD38⁻ while all of the MP subsets are CD34⁺CD38⁺. The HSC and MP cells are all negative for expression of antigens from a panel of lineage markers.

It is shown herein that decreased numbers of GMP (CD34⁺CD38⁺CD45RA⁺IL-3Rα^{lo}) relative to CMP (CD34⁺CD38⁺CD45RA⁻ IL-3Rα^{lo}) and MEP (CD34⁺CD38⁺CD45RA⁻IL-3Rα⁻) is diagnostic of MDS. The cut-off for diagnosis may be a decrease of at least about 1.5-fold, at least about 2-fold, or more.

Determination of the prognosis for a patient with established MDS to progress to overt leukemia, particularly AML, is indicated by increasing numbers of lineage-CD34+CD38+CD45RA+ cells, even when there is no definite morphologic evidence of increased blasts in bone marrow aspirate smears.

The methods of the invention may use any appropriate method to detect the presence of apparently expressed cell surface markers, including flow cytometry, immunohistochemistry, immunofluorescence, in situ hybridization, and the like on cytologic specimens or histologic sections of bone marrow, peripheral blood or other human tissue and body fluids. All or a subset of the indicated markers may be used for analysis, for example a combination of staining for CD34, CD38 and CD45RA can be sufficient for diagnosis.

Conventional cytogenetics can provide a test for diagnosing myelodysplasia;

however, a significant proportion of MDS cases do not harbor a readily detectable karyotypic abnormality at initial onset, and thus diagnosis may be delayed by months or years. The present invention provides a simple, rapid, and highly accurate method to diagnose myelodysplastic syndrome with high sensitivity and specificity. Moreover, the assay does not depend on culturing of cells or obtaining appropriate metaphase spreads, as is required for conventional cytogenetic analysis. Most importantly, the technique does not depend on the presence of cytogenetic abnormality to render a diagnosis.

Further benefits are provided by analysis of later stage MDS for the presence of antigenic changes that are prognostic of a tendency to progress to AML, and as a means of distinguishing MDS/AML from de novo AML. At present, there are no verified markers that can distinguish between de novo AML and AMUMDS. Although conventional cytogenetics and molecular testing by PCR may identify such patients, these tests require several days to be performed. Flow cytometry offers a rapid and easily accessible method for making this distinction.

“Diagnosis” as used herein generally includes determination of a subject's susceptibility to a disease or disorder, determination as to whether a subject is presently affected by a disease or disorder, prognosis of a subject affected by a disease or disorder (e.g., identification of cancerous states, stages of cancer, or responsiveness of cancer to therapy), and use of therametrics (e.g., monitoring a subject's condition to provide information as to the effect or efficacy of therapy).

The term “biological sample” encompasses a variety of sample types obtained from an organism and can be used in a diagnostic or monitoring assay. The term encompasses blood and other liquid samples of biological origin, solid tissue samples, such as a biopsy specimen or tissue cultures or cells derived therefrom and the progeny thereof. The term encompasses samples that have been manipulated in any way after their procurement, such as by treatment with reagents, solubilization, or enrichment for certain components. The term encompasses a clinical sample, and also includes cells in cell culture, cell supernatants, cell lysates, serum, plasma, biological fluids, and tissue samples.

The terms “treatment”, “treating”, “treat” and the like are used herein to generally refer to obtaining a desired pharmacologic and/or physiologic effect. The effect may be prophylactic in terms of completely or partially preventing a disease or symptom thereof and/or may be therapeutic in terms of a partial or complete stabilization or cure for a disease and/or adverse effect attributable to the disease. “Treatment” as used herein covers any treatment of a disease in a mammal, particularly a human, and includes: (a) preventing the disease or symptom from occurring in a subject which may be predisposed to the disease or symptom but has not yet been diagnosed as having it; (b) inhibiting the disease symptom, i.e., arresting its development; or (c) relieving the disease symptom, i.e., causing regression of the disease or symptom.

The terms “individual,” “subject,” “host,” and “patient,” used interchangeably herein and refer to any mammalian subject for whom diagnosis, treatment, or therapy is desired, particularly humans.

The terms “cancer”, “neoplasm”, “tumor”, and “carcinoma”, are used interchangeably herein to refer to cells which exhibit relatively autonomous growth, so that they exhibit an aberrant growth phenotype characterized by a significant loss of control of cell proliferation. In general, cells of interest for detection or treatment in the present application include precancerous (e.g., benign), malignant, pre-metastatic, metastatic, and non-metastatic cells. Detection of cancerous cells is of particular interest. The term “normal” as used in the context of “normal cell,” is meant to refer to a cell of an untransformed phenotype or exhibiting a morphology of a non-transformed cell of the tissue type being examined. “Cancerous phenotype” generally refers to any of a variety of biological phenomena that are characteristic of a cancerous cell, which phenomena can vary with the type of cancer. The cancerous phenotype is generally identified by abnormalities in, for example, cell growth or proliferation (e.g., uncontrolled growth or proliferation), regulation of the cell cycle, cell mobility, cell-cell interaction, or metastasis, etc.

“Therapeutic target” refers to a gene or gene product that, upon modulation of its activity (e.g., by modulation of expression, biological activity, and the like), can provide for modulation of the cancerous phenotype. As used throughout, “modulation” is meant to refer to an increase or a decrease in the indicated phenomenon (e.g., modulation of a biological activity refers to an increase in a biological activity or a decrease in a biological activity).

Myelodysplastic syndrome (MDS). The myelodysplastic syndromes (MDS) are classified according to features of cellular morphology, etiology, clinical presentation, and cytogenetic features. The morphological classification of the MDS is largely based on the percent of myeloblasts in the bone marrow and blood, the type and degree of myeloid dysplasia, and the presence of ringed sideroblasts. The clinical classification of the MDS depends upon whether there is an identifiable etiology and whether the MDS has been treated previously. Current classification of MDS cellular types and subtypes are listed below.

Included in MDS is Refractory anemia (RA). In patients with RA, the myeloid and megakaryocytic series in the bone marrow appear normal to conventional tests, but megaloblastoid erythroid hyperplasia is present. Dysplasia is usually minimal. Marrow blasts are less than 5%, and no peripheral blasts are present. Macrocytic anemia with reticulocytopenia is present in the blood. Transformation to acute leukemia is rare, and median survival varies from 2 years to 5 years in most series.

Refractory anemia with ringed sideroblasts (RARS). In patients with RARS, the blood and marrow are identical to those in patients with RA, except that at least 15% of marrow red cell precursors are ringed sideroblasts. Prognosis is similar to that of RA.

Refractory anemia with excess blasts (RAEB). In patients with RAEB, there is significant evidence of disordered myelopoiesis and megakaryocytopoiesis in addition to abnormal erythropoiesis. Because of differences in prognosis related to progression to a frank AML, this cellular classification is comprised of two categories, refractory anemia with excess blasts-1 (RAEB-1) and refractory anemia with excess blasts-2 (RAEB-2). Combined, the two categories account for approximately 40% of all patients with MDS. RAEB-1 is characterized by 5% to 9% blasts in the bone marrow and less than 5% blasts in the blood. Approximately 25% of cases of RAEB-1 progress to AML. Median survival is approximately 18 months. RAEB-2 is characterized by 10% to 19% blasts in the bone marrow. Approximately 33% of cases of RAEB-2 progress to AML. Median survival for RAEB-2 is approximately 10 months.

Refractory cytopenia with multilineage dysplasia (RCMD). In patients with RCMD, bicytopenia or pancytopenia is present. In addition, dysplastic changes are present in 10% or more of the cells in two or more myeloid cell lines. There are less than 1% blasts in the blood and less than 5% blasts in the bone marrow. Auer rods are not present. Monocytes in the blood are less than 1×10⁹. RCMD accounts for approximately 24% of cases of MDS. The frequency of evolution to acute leukemia is 11%. The overall median survival is 33 months. Refractory cytopenia with multilineage dysplasia and ringed sideroblasts (RCMD-RS) represents another category of RMDS. In RCMD-RS, features of RCMD are present, and more than 15% of erythroid precursors in the bone marrow are ringed sideroblasts. RCMD-RS accounts for approximately 15% of cases of MDS. Survival in RCMD-RS is similar to that in primary RCMD.

Myelodysplastic syndrome associated with an isolated del(5q) chromosome abnormality is associated with an isolated del(5q) cytogenetic abnormality. Blasts in both blood and bone marrow are less than 5%. This subtype is associated with a long survival.

The mainstay of treatment of the myelodysplastic syndromes (MDS) has traditionally been supportive care. Anemia is treated with red cell transfusions regularly, and patients receiving chronic red cell transfusions should be considered for iron chelation therapy with subcutaneously administered desferrioxamine and vitamin C or oral deferasirox. The use of erythropoietin may improve anemia, although effective treatment may require substantially higher doses of erythropoietin than are used for other indications (150-300 μg/kg/day).

Although therapy with cytotoxic agents has occasionally been beneficial, results are usually disappointing, and responses are often brief when achieved. Hematologic improvement induced by therapy may be offset by adverse changes in quality of life. Autologous bone marrow or peripheral blood progenitor cell transplantation is under clinical evaluation for subsets of patients who achieve remission following cytotoxic remission induction therapy.

Acute Myelocytic Leukemia (AML, Acute Myelogenous Leukemia; Acute Myeloid Leukemia). In AML, malignant transformation and uncontrolled proliferation of an abnormally differentiated, long-lived myeloid progenitor cell results in high circulating numbers of immature blood forms and replacement of normal marrow by malignant cells. Symptoms include fatigue, pallor, easy bruising and bleeding, fever, and infection; symptoms of leukemic infiltration are present in only about 5% of patients (often as skin manifestations). Examination of peripheral blood smear and bone marrow is diagnostic. Treatment includes induction chemotherapy to achieve remission and post-remission chemotherapy (with or without stem cell transplantation) to avoid relapse.

AML has a number of subtypes that are distinguished from each other by morphology, immunophenotype, cytochemistry, and cytogeneic features. Five classes are described, based on predominant cell type, including myeloid, myeloid-monocytic, monocytic, erythroid, and megakaryocytic. Acute promyelocytic leukemia is a particularly important subtype, representing 10 to 15% of all cases of AML, striking a younger age group (median age 31 yr) and particular ethnicity (Hispanics), in which the patient commonly presents with a coagulation disorder.

Remission induction rates range from 50 to 85%. Long-term disease-free survival reportedly occurs in 20 to 40% of patients and increases to 40 to 50% in younger patients treated with stem cell transplantation.

Prognostic factors help determine treatment protocol and intensity; patients with strongly negative prognostic features are usually given more intense forms of therapy, because the potential benefits are thought to justify the increased treatment toxicity. The most important prognostic factor is the leukemia cell karyotype; favorable karyotypes include t(15;17), t(8;21), and inv16 (p13;q22). Negative factors include increasing age, a preceding myelodysplastic phase, secondary leukemia, high WBC count, and absence of Auer rods. The FAB or WHO classification alone does not predict response.

Initial therapy attempts to induce remission and differs most from ALL in that AML responds to fewer drugs. The basic induction regimen includes cytarabine by continuous IV infusion or high doses for 5 to 7 days; daunorubicin or idarubicin is given IV for 3 days during this time. Some regimens include 6-thioguanine, etoposide, vincristine, and prednisone, but their contribution is unclear. Treatment usually results in significant myelosuppression, with infection or bleeding; there is significant latency before marrow recovery. During this time, meticulous preventive and supportive care is vital.

Stem and Progenitor Subsets. Bone marrow contains a number of hematopoietic stem and progenitor cell classes. The earliest cell in the lineage is the stem cell, or HSC. HSCs are functionally defined by their unique capacity to self-renew and to differentiate to produce all mature blood cell types. The earliest known lymphoid-restricted cell in adult mouse bone marrow is the common lymphocyte progenitor (CLP), and the earliest known myeloid-restricted cell is the common myeloid progenitor (CMP). A complete description of these cell subsets may be found in Akashi et al. (2000) Nature 404(6774):193, U.S. Pat. No. 6,465,247; and published application U.S. Ser. No. 09/956,279 (common myeloid progenitor); Kondo et al. (1997) Cell 91(5):661-7, and International application WO99/10478 (common lymphoid progenitor); and is reviewed by Kondo et al. (2003) Annu Rev Immunol. 21:759-806, each of which is herein specifically incorporated by reference.

CD34+ cells harbor virtually all in vitro clonogenic potential; however, the CD34+ population is heterogeneous. Only a small fraction (1-10%) of CD34+ cells that do not express mature lineage markers (Lin⁻, including the markers CD3, CD4, CD8, CD19, CD20, CD56, CD11b, CD14, and CD15) have multilineage (lymphoid and myeloid) developmental potential. The majority of CD34+ cells (90-99%) coexpress the CD38 antigen, and this subset contains most of the lineage-restricted progenitors.

In the myeloid lineage are three cell populations, termed CMPs, GMPs, and MEPs. These cells are CD34⁺CD38⁺, they are negative for multiple mature lineage markers including early lymphoid markers such as CD7, CD10, and IL-7R, and they are further distinguished by the markers CD45RA, an isoform of CD45 that can negatively regulate at least some classes of cytokine receptor signaling, and IL-3R. These characteristics are CD45RA⁻ IL-3Rα^{lo} (CMPs), CD45RA⁺IL-3Rα^{lo} (GMPs), and CD45RA⁻ IL-3Rα⁻ (MEPs). CD45RA⁻ IL-3Rα^{lo} cells give rise to GMPs and MEPs and at least one third generate both GM and MegE colonies on a single-cell level. All three of the myeloid lineage progenitors stain negatively for the markers Thy-1 (CD90), IL-7Rα (CD127); and with a panel of lineage markers, which lineage markers may include CD2; CD3; CD4; CD7; CD8; CD10; CD11b; CD14; CD19; CD20; CD56; and glycophorin A (GPA).

### Methods Of Analysis

Samples containing white blood cells, particularly including bone marrow samples, are stained with reagents specific for markers present on hematopoietic stem and progenitor cells, which markers are sufficient to distinguish the major stem and progenitor groups. The reagents, e.g. antibodies, may be detectably labeled, or may be indirectly labeled in the staining procedure.

In one embodiment of the invention, a biological sample from a patient suspected of

MDS is contacted with detectable labeled reagents specific for CD34; CD38; and CD45RA. Optional markers of interest include IL-3Rα; and lineage panel. The analysis of staining patterns in the CD34+ subset of hematopoietic cells provides the relative distribution of progenitor cells, which distribution diagnoses the presence of MDS. As described above, a decrease in the GMP populations relative to the total pool of myeloid progenitors indicates MDS, while an increase in cells having a GMP phenotype is indicative of a later disease progression to a leukemic state. The patient sample is compared to a control, usually a positive control and a negative control (or a standard test value). In another embodiment, the patient sample is compared to one or more time points through the course of the disease.

In addition to the markers useful in determining the distribution of progenitor cells, reagents selective for cell surface markers aberrantly expressed on HSC present in MDS and MDS/AML may also be used. Such markers include CD117, and one or more markers set forth in Table 1.

A marker combination of interest may include CD34 and CD38, which distinguishes hematopoietic stem cells, (CD34⁺, CD38⁻) from myeloid progenitor cells, which are (CD34⁺, CD38³⁰ ). The inclusion of CD45RA and IL-3Rα is also of interest because it allows a distinction between the three known myeloid progenitor cell subsets. Lineage panels may be included to distinguish progenitor cells from lineage committed cells, for example a panel including one or more of CD2; CD3; CD4; CD7; CD8; CD10; CD11 b; CD14; CD19; CD20; CD56; and glycophorin A.

The information thus derived is useful in prognosis and diagnosis, including susceptibility to acceleration of disease, status of a diseased state and response to changes in the environment, such as the passage of time, treatment with drugs or other modalities.

Additionally, MDS/HSC, which are cells having the phenotype of HSC, i.e. CD34⁺CD38⁻, and optionally one or more of CD90⁺, CD117⁺, lin⁻, and which aberrantly express one or more of the markers set forth in Table 1, can be ablated from patient samples, or isolated for research purposes, analyzed as to their ability to respond to therapeutic agents and treatments, screened for gene expression, and the like. The clinical samples can be further characterized by genetic analysis, proteomics, cell surface staining, or other means, in order to determine the presence of markers that are useful in classification.

Clinical samples for use in the methods of the invention may be obtained from a variety of sources, particularly bone marrow, although in some instances samples such as blood, lymph, cerebrospinal fluid, synovial fluid, and the like may be used. Such samples can be separated by centrifugation, elutriation, density gradient separation, apheresis, affinity selection, panning, FACS, centrifugation with Hypaque, etc. prior to analysis, and usually a mononuclear fraction (PBMC) will be used. Once a sample is obtained, it can be used directly, frozen, or maintained in appropriate culture medium for short periods of time. Various media can be employed to maintain cells. The samples may be obtained by any convenient procedure, such as the drawing of blood, venipuncture, biopsy, or the like. Usually a sample will comprise at least about 10² cells, more usually at least about 10³ cells, and preferable 10⁴, 10⁵ or more cells. Typically the samples will be from human patients, although animal models may find use, e.g. equine, bovine, porcine, canine, feline, rodent, e.g. mice, rats, hamster, primate, etc.

An appropriate solution may be used for dispersion or suspension of the cell sample. Such solution will generally be a balanced salt solution, e.g. normal saline, PBS, Hank's balanced salt solution, etc., conveniently supplemented with fetal calf serum or other naturally occurring factors, in conjunction with an acceptable buffer at low concentration, generally from 5-25 mM. Convenient buffers include HEPES, phosphate buffers, lactate buffers, etc.

Analysis of the cell staining will use conventional methods. Techniques providing accurate enumeration include fluorescence activated cell sorters, which can have varying degrees of sophistication, such as multiple color channels, low angle and obtuse light scattering detecting channels, impedance channels, etc. The cells may be selected against dead cells by employing dyes associated with dead cells (e.g. propidium iodide).

The affinity reagents may be specific receptors or ligands for the cell surface molecules indicated above. In addition to antibody reagents, peptide-MHC antigen and T cell receptor pairs may be used; peptide ligands and receptor; effector and receptor molecules, and the like. Antibodies and T cell receptors may be monoclonal or polyclonal, and may be produced by transgenic animals, immunized animals, immortalized human or animal B-cells, cells transfected with DNA vectors encoding the antibody or T cell receptor, etc. The details of the preparation of antibodies and their suitability for use as specific binding members are well-known to those skilled in the art.

Of particular interest is the use of antibodies as affinity reagents. Conveniently, these antibodies are conjugated with a label for use in separation. Labels include magnetic beads, which allow for direct separation, biotin, which can be removed with avidin or streptavidin bound to a support, fluorochromes, which can be used with a fluorescence activated cell sorter, or the like, to allow for ease of separation of the particular cell type. Fluorochromes that find use include phycobiliproteins, e.g. phycoerythrin and allophycocyanins, fluorescein and Texas red. Frequently each antibody is labeled with a different fluorochrome, to permit independent sorting for each marker.

The antibodies are added to a suspension of cells, and incubated for a period of time sufficient to bind the available cell surface antigens. The incubation will usually be at least about 5 minutes and usually less than about 30 minutes. It is desirable to have a sufficient concentration of antibodies in the reaction mixture, such that the efficiency of the separation is not limited by lack of antibody. The appropriate concentration is determined by titration. The medium in which the cells are separated will be any medium that maintains the viability of the cells. A preferred medium is phosphate buffered saline containing from 0.1 to 0.5% BSA. Various media are commercially available and may be used according to the nature of the cells, including Dulbecco's Modified Eagle Medium (dMEM), Hank's Basic Salt Solution (HBSS), Dulbecco's phosphate buffered saline (dPBS), RPMI, Iscove's medium, PBS with 5 mM EDTA, etc., frequently supplemented with fetal calf serum, BSA, HSA, etc.

The labeled cells are then quantitated as to the expression of cell surface markers as previously described. It is particularly convenient in a clinical setting to perform the immunoassay in a self-contained apparatus. A number of such methods are known in the art. The apparatus will generally employ a continuous flow-path of a suitable filter or membrane, having at least three regions, a fluid transport region, a sample region, and a measuring region. The sample region is prevented from fluid transfer contact with the other portions of the flow path prior to receiving the sample. After the sample region receives the sample, it is brought into fluid transfer relationship with the other regions, and the fluid transfer region contacted with fluid to permit a reagent solution to pass through the sample region and into the measuring region. The measuring region may have bound to it a conjugate of an enzyme with progenitor cell specific antibodies.

The comparison of a differential progenitor analysis obtained from a patient sample, and a reference differential progenitor analysis is accomplished by the use of suitable deduction protocols, Al systems, statistical comparisons, etc. A comparison with a reference differential progenitor analysis from normal cells, cells from similarly diseased tissue, and the like, can provide an indication of the disease staging. A database of reference differential progenitor analyses can be compiled. An analysis of particular interest tracks a patient, e.g. in the initial disease presentation and monitoring thereafter, such that acceleration of disease is observed at an early stage. The methods of the invention provide detection of acceleration prior to onset of clinical symptoms, and therefore allow early therapeutic intervention, e.g. initiation of chemotherapy, increase of chemotherapy dose, changing selection of chemotherapeutic drug, and the like.

Kits may be provided, where the kit will comprise a staining reagents that are sufficient to differentially identify the CD34⁺ stem and progenitor subsets described herein. A marker combination of interest may include CD34 and CD38, CD45RA and IL-3Rα. Lineage panels may be included to distinguish progenitor cells from lineage committed cells. Also of interest are reagents specific for the markers set forth in Table 1. The staining reagents are preferably antibodies, and may be detectably labeled. Kits may also include tubes, buffers, etc., and instructions for use.

### Treatment of Cancer

The invention further provides methods for treating MDS, by specifically targeting or ablating hematopoietic stem cells having an aberrant phenotype (HSC/MDS) characterized by overexpression of one or more markers set forth in Table 1. The methods provide for decreasing the number of HSC/MDS cells bearing a specific marker or combination of markers, as provided herein, decreasing expression of a gene that is differentially expressed in a HSC/MDS cell. In general, the methods comprise contacting a HSC/MDS cell with a binding agent, e.g. an antibody or ligand specific for a marker or combination of markers provided herein.

Methods for treating MDS generally comprise administering to an individual in need thereof a substance that reduces HSC/MDS cell growth. Whether a substance, or a specific amount of the substance, is effective in treatment can be assessed using any of a variety of known diagnostic assays, including, but not limited to biopsy, contrast radiographic studies, CAT scan, etc. The substance can be administered systemically or locally, usually systemically.

HSC/MDS expressing a marker or combination of markers of the present invention are also useful for in vitro assays and screening to detect factors and chemotherapeutic agents that are active on HSC/MDS cells. Of particular interest are screening assays for agents that are active on human cells. A wide variety of assays may be used for this purpose, including immunoassays for protein binding; determination of cell growth, differentiation and functional activity; production of factors; and the like. In other embodiments, isolated polypeptides corresponding to a marker or combination of markers of the present invention are useful in drug screening assays.

In screening assays for biologically active agents, anti-proliferative drugs, etc. the HSC/MDS composition is contacted with the agent of interest, and the effect of the agent assessed by monitoring output parameters on cells, such as expression of markers, cell viability, and the like; or binding efficacy or effect on enzymatic or receptor activity for polypeptides. The cells may be freshly isolated, cultured, genetically altered, or transplanted as xenografts, and the like. The cells may be environmentally induced variants of clonal cultures: e.g. split into independent cultures and grown under distinct conditions, for example with or without drugs; in the presence or absence of cytokines or combinations thereof. The manner in which cells respond to an agent, particularly a pharmacologic agent, including the timing of responses, is an important reflection of the physiologic state of the cell.

Parameters are quantifiable components of cells, particularly components that can be accurately measured, desirably in a high throughput system. A parameter can be any cell component or cell product including cell surface determinant, receptor, protein or conformational or posttranslational modification thereof, lipid, carbohydrate, organic or inorganic molecule, nucleic acid, e.g. mRNA, DNA, etc. or a portion derived from such a cell component or combinations thereof. While most parameters will provide a quantitative readout, in some instances a semi-quantitative or qualitative result will be acceptable. Readouts may include a single determined value, or may include mean, median value or the variance, etc. Characteristically a range of parameter readout values will be obtained for each parameter from a multiplicity of the same assays. Variability is expected and a range of values for each of the set of test parameters will be obtained using standard statistical methods with a common statistical method used to provide single values.

Agents of interest for screening include known and unknown compounds that encompass numerous chemical classes, primarily organic molecules, which may include organometallic molecules, inorganic molecules, genetic sequences, etc. An important aspect of the invention is to evaluate candidate drugs, including toxicity testing; and the like.

In addition to complex biological agents candidate agents include organic molecules comprising functional groups necessary for structural interactions, particularly hydrogen bonding, and typically include at least an amine, carbonyl, hydroxyl or carboxyl group, frequently at least two of the functional chemical groups. The candidate agents often comprise cyclical carbon or heterocyclic structures and/or aromatic or polyaromatic structures substituted with one or more of the above functional groups. Candidate agents are also found among biomolecules, including peptides, polynucleotides, saccharides, fatty acids, steroids, purines, pyrimidines, derivatives, structural analogs or combinations thereof.

Included are pharmacologically active drugs, genetically active molecules, etc.

Compounds of interest include chemotherapeutic agents, hormones or hormone antagonists, etc. Exemplary of pharmaceutical agents suitable for this invention are those described in, “The Pharmacological Basis of Therapeutics,” Goodman and Gilman, McGraw-Hill, New York, N.Y., (1996), Ninth edition, under the sections: Water, Salts and Ions; Drugs Affecting Renal Function and Electrolyte Metabolism; Drugs Affecting Gastrointestinal Function; Chemotherapy of Microbial Diseases; Chemotherapy of Neoplastic Diseases; Drugs Acting on Blood-Forming organs; Hormones and Hormone Antagonists; Vitamins, Dermatology; and Toxicology, all incorporated herein by reference. Also included are toxins, and biological and chemical warfare agents, for example see Somani, S.M. (Ed.), “Chemical Warfare Agents,” Academic Press, New York, 1992).

Test compounds include all of the classes of molecules described above, and may further comprise samples of unknown content. Of interest are complex mixtures of naturally occurring compounds derived from natural sources such as plants. While many samples will comprise compounds in solution, solid samples that can be dissolved in a suitable solvent may also be assayed. Samples of interest include environmental samples, e.g. ground water, sea water, mining waste, etc.; biological samples, e.g. lysates prepared from crops, tissue samples, etc.; manufacturing samples, e.g. time course during preparation of pharmaceuticals; as well as libraries of compounds prepared for analysis; and the like. Samples of interest include compounds being assessed for potential therapeutic value, i.e. drug candidates.

The term “samples” also includes the fluids described above to which additional components have been added, for example components that affect the ionic strength, pH, total protein concentration, etc. In addition, the samples may be treated to achieve at least partial fractionation or concentration. Biological samples may be stored if care is taken to reduce degradation of the compound, e.g. under nitrogen, frozen, or a combination thereof. The volume of sample used is sufficient to allow for measurable detection, usually from about 0.1 to 1 ml of a biological sample is sufficient.

Compounds, including candidate agents, are obtained from a wide variety of sources including libraries of synthetic or natural compounds. For example, numerous means are available for random and directed synthesis of a wide variety of organic compounds, including biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs.

Agents are screened for biological activity by adding the agent to at least one and usually a plurality of cell samples, usually in conjunction with cells lacking the agent. The change in parameters in response to the agent is measured, and the result evaluated by comparison to reference cultures, e.g. in the presence and absence of the agent, obtained with other agents, etc.

The agents are conveniently added in solution, or readily soluble form, to the medium of cells in culture. The agents may be added in a flow-through system, as a stream, intermittent or continuous, or alternatively, adding a bolus of the compound, singly or incrementally, to an otherwise static solution. In a flow-through system, two fluids are used, where one is a physiologically neutral solution, and the other is the same solution with the test compound added. The first fluid is passed over the cells, followed by the second. In a single solution method, a bolus of the test compound is added to the volume of medium surrounding the cells. The overall concentrations of the components of the culture medium should not change significantly with the addition of the bolus, or between the two solutions in a flow through method.

Preferred agent formulations do not include additional components, such as preservatives, that may have a significant effect on the overall formulation. Thus preferred formulations consist essentially of a biologically active compound and a physiologically acceptable carrier, e.g. water, ethanol, DMSO, etc. However, if a compound is liquid without a solvent, the formulation may consist essentially of the compound itself.

A plurality of assays may be run in parallel with different agent concentrations to obtain a differential response to the various concentrations. As known in the art, determining the effective concentration of an agent typically uses a range of concentrations resulting from 1:10, or other log scale, dilutions. The concentrations may be further refined with a second series of dilutions, if necessary. Typically, one of these concentrations serves as a negative control, i.e. at zero concentration or below the level of detection of the agent or at or below the concentration of agent that does not give a detectable change in the phenotype.

Various methods can be utilized for quantifying the presence of the selected markers. For measuring the amount of a molecule that is present, a convenient method is to label a molecule with a detectable moiety, which may be fluorescent, luminescent, radioactive, enzymatically active, etc., particularly a molecule specific for binding to the parameter with high affinity. Fluorescent moieties are readily available for labeling virtually any biomolecule, structure, or cell type. Immunofluorescent moieties can be directed to bind not only to specific proteins but also specific conformations, cleavage products, or site modifications like phosphorylation. Individual peptides and proteins can be engineered to autofluoresce, e.g. by expressing them as green fluorescent protein chimeras inside cells (for a review see Jones et al. (1999) Trends Biotechnol. 17(12):477-81). Thus, antibodies can be genetically modified to provide a fluorescent dye as part of their structure. Depending upon the label chosen, parameters may be measured using other than fluorescent labels, using such immunoassay techniques as radioimmunoassay (RIA) or enzyme linked immunosorbance assay (ELISA), homogeneous enzyme immunoassays, and related non-enzymatic techniques. The quantitation of nucleic acids, especially messenger RNAs, is also of interest as a parameter. These can be measured by hybridization techniques that depend on the sequence of nucleic acid nucleotides. Techniques include polymerase chain reaction methods as well as gene array techniques. See Current Protocols in Molecular Biology, Ausubel et al., eds, John Wiley & Sons, New York, N.Y., 2000; Freeman et al. (1999) Biotechniques 26(1):112-225; Kawamoto et al. (1999) Genome Res 9(12):1305-12; and Chen et al. (1998) Genomics 51(3):313-24, for examples.

### Depletion of HSC/MDS

Depletion of HSC/MDS is useful in the treatment of MDS. Depletion can be achieved by several methods. Depletion is defined as a reduction in the target population by up to about 30%, or up to about 40%, or up to about 50%, or up to about 75% or more. An effective depletion is usually determined by the sensitivity of the particular disease condition to the levels of the target population. Thus in the treatment of certain conditions a depletion of even about 20% could be beneficial.

A marker-specific agent that specifically depletes the targeted HSC/MDS is used to contact the patient blood or bone marrow in vitro or in vivo, wherein after the contacting step, there is a reduction in the number of viable HSC/MDS in the targeted population. An exemplary agent for such purposes is an antibody that specifically binds to a marker or combination of markers of the present invention on the surface of the targeted HSC/MDS. An effective dose of antibodies for such a purpose is sufficient to decrease the targeted population to the desired level, for example as described above. Antibodies for such purposes may have low antigenicity in humans or may be humanized antibodies.

In one embodiment of the invention, antibodies for depleting target population are added to patient blood or bone marrow in vivo. In another embodiment, the antibodies are added to the patient blood or bone marrow ex vivo. Beads coated with the antibody of interest can be added to the blood, target cells bound to these beads can then be removed from the blood using procedures common in the art. In one embodiment the beads are magnetic and are removed using a magnet. Alternatively, when the antibody is biotinylated, it is also possible to indirectly immobilize the antibody onto a solid phase which has adsorbed avidin, streptavidin, or the like. The solid phase, usually agarose or sepharose beads are separated from the blood or bone marrow by brief centrifugation. Multiple methods for tagging antibodies and removing such antibodies and any cells bound to the antibodies are routine in the art. Once the desired degree of depletion has been achieved, the blood or bone marrow is returned to the patient. Depletion of target cells ex vivo decreases the side effects such as infusion reactions associated with the intravenous administration. An additional advantage is that the repertoire of available antibodies is expanded significantly as this procedure does not have to be limited to antibodies with low antigenicity in humans or humanized antibodies.

It is to be understood that this invention is not limited to the particular methodology, protocols, cell lines, animal species or genera, and reagents described, as such may vary. It is also to be understood that the terminology used herein is for the purpose of describing particular embodiments only, and is not intended to limit the scope of the present invention, which will be limited only by the appended claims.

As used herein the singular forms “a”, “and”, and “the” include plural referents unless the context clearly dictates otherwise. Thus, for example, reference to “a cell” includes a plurality of such cells and reference to “the culture” includes reference to one or more cultures and equivalents thereof known to those skilled in the art, and so forth. All technical and scientific terms used herein have the same meaning as commonly understood to one of ordinary skill in the art to which this invention belongs unless clearly indicated otherwise.

### Experimental

### EXAMPLE 1

### Myelodysplastic Syndromes are Characterized by Gene Expression Changes in Hematopoietic Stem Cells and Alterations in Hematopoietic Stem Cell and Myeloid Progenitor Composition

The myelodysplastic syndromes (MDS) represent a heterogeneous group of disorders characterized by peripheral cytopenias due to impaired hematopoietic differentiation. To date, most data characterizing the immature hematopoietic compartment in MDS have relied on evaluation of CD34+ bone marrow cells, which are a heterogeneous population containing a predominance of oligo- and unilineage-potent progenitors and few hematopoietic stem cells (HSC).

In this study we show that MDS are disorders of HSC, evidenced by the presence of recurrent cytogenetic alterations, including -5q, -7, and -20q, in highly purified HSC (Lin−CD34+CD38-CD9O+CD45RA−) by FISH. Because MDS HSC harbor cytogenetic changes, we sought to better characterize the molecular basis of MDS HSC function by performing whole transcriptome analysis of highly purified HSC and committed myeloid progenitor populations from low-risk (n=8) and high-risk (n=2) MDS patients. When compared to control HSC from healthy patients (n=10), MDS HSC showed broad transcriptional changes.

Using the significance analysis of microarrays (SAM) algorithm and Ingenuity Pathways Analysis software, we identified 3,258 differentially expressed genes (FDR<0.1) with increased expression of genes positively associated with cell growth and proliferation (p<0.001) and increased expression of inflammatory response genes (p<0.015). Interestingly, while MDS common myeloid progenitors (CMP, Lin−CD34+CD38+CD123+CD45RA−) showed increased expression of cell death-related genes when compared to normal CMP (p<0.001), neither MDS HSC nor multipotent progenitors (MPP, Lin−CD34+CD38-CD9O-CD45RA−) showed significant differential expression of these genes when compared to their normal counterparts.

To assess the cellular and developmental correlates of HSC/committed progenitor transcriptional changes, we evaluated by flow cytometry the frequency of HSC and committed myeloid progenitors in bone marrow aspirates from 35 low-risk MDS, 6 high-risk MDS and 32 healthy patient samples (range 4-84 yo). Low-risk MDS bone marrow samples showed significantly increased numbers of HSC compared to normal bone marrow samples (+3-fold change, p<0.03). In addition, myeloid progenitor composition was frequently altered in low-risk MDS patients, with decreased percentages of granulocyte-macrophage progenitors (GMP, Lin−CD34+CD38+CD123+CD45RA+) when expressed as a percentage of total myeloid progenitors [including GMP, CMP and megakaryocyte-erythroid progenitors (MEP, Lin−CD34+CD38+CD123IoCD45RA−)] (−2.3-fold change, p<1e-6). This altered myeloid progenitor profile was highly specific to MDS, even when MDS patient samples were compared to a group of control bone marrow samples from non-MDS patients exhibiting at least one cytopenia (n=34, p<1e-5), allowing for the distinction of MDS samples from non-MDS cytopenias with 0.89 sensitivity and 0.89 specificity.

Together, these data indicate that MDS HSC exhibit significantly altered gene expression profiles and suggest that gene expression changes in MDS HSC induce the altered developmental fate decisions and transcriptional changes observed in MDS committed myeloid progenitors. These data also demonstrate that the changes in MDS myeloid progenitor composition provide a novel, flow cytometric method for distinguishing MDS from other hematologic conditions that mimic MDS. Finally, these studies indicate that molecular characterization of MDS phenotypes may require evaluation of purified hematopoietic progenitors in order to account for the differential effect of MDS-associated changes on specific hematopoietic progenitor populations.

All publications and patent applications mentioned in this specification are herein incorporated by reference to the same extent as if each individual publication or patent application was specifically and individually indicated to be incorporated by reference.

The invention now being fully described, it will be apparent to one of ordinary skill in the art that many changes and modifications can be made thereto without departing from the spirit or scope of the appended claims.

1. A method of phenotyping a myelodysplastic condition, the method comprising:
combining a hematologic sample from a patient suspected of said myelodysplastic condition with specific binding members that are sufficient to distinguish the distribution of cells with hematopoietic stem and progenitor subsets;
determining the distribution of progenitor cells between said subsets,
wherein the distribution of progenitor cells is indicative of the phenotype of said myelodysplastic condition.

2. The method according to claim 1, wherein said hematopoietic stem and progenitor subsets include one or more of HSC, CMP, MEP and GMP.

3. The method according to claim 1, wherein said specific binding members are antibodies.

4. The method according to claim 3, wherein said antibodies include specificities for CD34 and CD38.

5. The method according to claim 4, wherein said antibodies further include specificities for CD45RA.

6. The method according to claim 5, further comprising antibodies specific for a lineage panel.

7. The method of claim 1, wherein said hematologic sample is a bone marrow sample.

8. The method of claim 1, wherein a decrease in the fraction of myeloid progenitor cells that are granulocyte macrophage progenitor cells is indicative of an initial diagnosis of MDS.

9. The method of claim 1, wherein said sample is obtained from a patient diagnosed with ongoing MDS, wherein in increase in the fraction of myeloid progenitor cells having a granulocyte macrophage progenitor cell phenotype is indicative of a prognosis to progress to acute myelogenous leukemia.

10. The method of claim 1, wherein said markers further comprise a specific binding member for at least one marker set forth in Table 1.

11. The method of claim 10, further comprising selected for HSC/MDS on the basis of aberrant expression of said at least one marker from Table 1.

12. An isolated HSC/MDS obtained by the method set forth in claim 10.

13. A method of screening a candidate chemotherapeutic agent for effectiveness against an HSC/MDS, the method comprising:
contacting said agent with the cell composition of claim 12, and
determining the effectiveness of said agent against said HSC/MDS.

14. A method of targeting or depleting HSC/MDS cells, the method comprising contacting a population of hematopoietic stem cells with an agent that specifically binds a marker or combination of markers set forth in Table 1.

15. A kit for use in any of the methods set forth in claim 1.
